(19) United States
(12) Patent Application Publication
(10) Pub. No.: US 2011/0088594 A1
(43) Pub. Date: Apr. 21, 2011

(54) FLUOROSURFACTANTS

(76) Inventors: Eckhard Claus, Frankfurt Am Main (DE); Fanny Schooren, Muehltal (DE); Martin Seidel, Darmstadt (DE); Wolfgang Hierse, Gross-Zimmern (DE); Peer Kirsch, Kanagawa (JP)

(21) Appl. No.: 12/997,356

(22) PCT Filed: May 15, 2009
(86) PCT No.: PCT/EP09/03487

§ 371 Date: Dec. 10, 2010

(30) Foreign Application Priority Data
Jun. 12, 2008 (DE) 102008027.7

(51) Int. Cl. C09D 7/12; C07C 311/16; C07C 69/76; C07C 67/14; C07C 67/08

(52) U.S. Cl. 106/287.25; 560/12; 560/111; 106/287.24

(57) ABSTRACT

The present invention relates to compounds of the formulae (I), (II) and (III), to the use of compounds containing at least one perfluoro-tert-butoxy group and at least one substituted or unsubstituted aromatic end group as surface-active agent, and to a process for the preparation of compounds of the formula (II).

The present invention relates to compounds of the formulae (I), (II) and (III), to the use of compounds containing at least one perfluoro-tert-butoxy group and at least one substituted or unsubstituted aromatic end group as surface-active agent, and to a process for the preparation of compounds of the formula (II).

Fluorosurfactants have an outstanding ability to lower the surface tension, which is utilised, for example, in the hydrophobicisation of surfaces, for example of textiles, paper, glass, building materials or adsorbents. In addition, it is possible for them to be used as interface promoter or emulsifier or viscosity reducer in paints, coatings or adhesives.

In general, fluorosurfactants contain perfluoroalkyl substituents, which are broken down in the environment by biological and/or other oxidation processes to give perfluoroalkylcarboxylic acids (PFCAs) and -sulfonic acids (PFASs). In recent years, the accumulation of perfluoroalkylcarboxylic acids (PFCAs) and perfluoroalkylsulfonic acids (PFASs) in nature has given cause for concern. PFCAs and PFASs are highly persistent compounds, whose long-chain variants (containing perfluoroalkyl chains having 8 or more carbon atoms) have a bioaccumulative potential. They are in some cases suspected of causing health damage (G. L. Kennedy, Jr., J. L. Butenhoff, G. W. Olsen, J. C. O'Connor, A. M. Seacat, R. G. Biegel, S. R. Murphy, D. G. Farrar, Critical Review in Toxicology, 2004, 34, 351-384).

U.S. Pat. No. 3,668,233 and U.S. Pat. No. 3,385,904 describe perfluorinated tertiary alcohols and some derivatives of these alcohols.

Omnova markets polymers whose side chains contain terminal CF₃ or C₂F₅ groups. The international patent application WO 03/010128 describes perfluoroalkyl-substituted amines, acids, amino acids and thioether acids which contain a C3-20-perfluoroalkyl group.

JP-A-2001/133984 discloses surface-active compounds containing perfluoroalkoxy chains which are suitable for use in antireflection coatings. JP-A-09/111,286 discloses the use of perfluoropolyether surfactants in emulsions.

The earlier German patent application DE 102005000858 describes compounds which carry at least one terminal pentafluorosulfuranyl group or at least one terminal trifluoromethoxy group and contain a polar end group, are surface-active and are suitable as surfactants.

There continues to be a demand for alternative surface-active substances, preferably having a property profile comparable to classical fluorosurfactants and having equally good chemical versatility, which preferably do not break down to give long-chain persistent fluorocarboxylic or fluorosulfonic acids on oxidative or reductive degradation or are preferably effective in significantly lower doses than conventional fluorosurfactants.

The object of the present invention is therefore to provide novel fluorinated compounds which preferably have one or more of the said advantageous properties.

This object is achieved by the compounds of the formula (I) according to the invention. It has furthermore been found that compounds which contain at least one perfluoro-tert-butoxy group and at least one substituted or unsubstituted aromatic end group are suitable as surface-active agents.

The present invention therefore relates firstly to compounds of the formula I

[Yₙ-L-Xₘ]ₒ—Ar (I)

where

### Y═(CF₃)₃C—O,

L=linear or branched alkyl having at least 2 C atoms, where one or more non-adjacent C atoms may be replaced by O, S and/or N, and/or one or more double and/or triple bonds may be present in the chain and/or side chain,

### X═O(O)C, NH(O)C, O, S, N, OSO₂

Ar=an unsubstituted or mono- or polysubstituted aromatic ring or condensed ring system having 6 to 18 C atoms, optionally containing N, O and/or S,
n=1-3, preferably 1,
m=0 or 1, preferably 1, and
o=1-3, preferably 1-2,
where, for o≠1, different L and X may in each case be present and (nonafluoro-tert-butoxy)ethyl tosylate and di(perfluoro-tert-butoxyethyl) phthalate are excluded.

Ar is preferably an aromatic ring or condensed ring system which is mono- or polysubstituted by halogen, alkyl, cycloalkyl, aryl, —OH, —OR¹, —NR¹R², —C(O)OH, —COOR¹, —CONR¹R² or —SO₂NR¹R², where R¹ and R²=independently of one another H, linear or branched alkyl, cycloalkyl or alkylaryl.

X is preferably O(O)C.

L is preferably linear alkyl.

The compounds according to the invention may contain one or more perfluoro-tert-butoxy groups, preferably one, two or three such groups. Compounds containing one perfluoro-tert-butoxy group are particularly preferred.

Besides the perfluoro-tert-butoxy group(s), the compounds according to the invention preferably contain no further fluorinated groups.

A preferred variant of the invention is compounds of the formula (II)

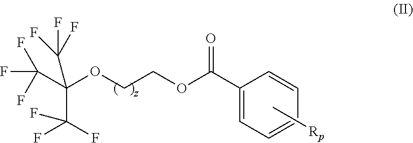

where
z=1-30, preferably z=1-8,
R═H, C1-C8-alkyl, —OH, —OR¹, —NR¹R², —SO₂NR¹R²,
R¹ and R²=independently of one another H, C1-C8-alkyl and
p=0-3, preferably 1.

Particular preference is given to compounds where z=1-4, in particular z=1, p=1 and R═H, —CH₂—CH₂—CH₃, —OH, —OEt, —NMe₂, —SO₂N(CH₂CH₃)₂, —SO₂N(CH₂CH₂CH₃)₂.

In a further variant of the invention, compounds containing the aromatic substituents in the para-position are preferred. However, it is also possible to employ compounds containing the aromatic substituents in the ortho- or meta-position, and a combination of substituents in the ortho-, meta- and para-position.

Particularly preferred compounds of the present invention are the compounds of the formula (III)

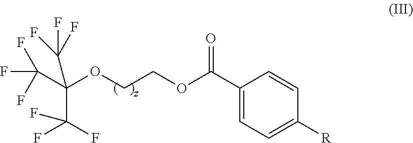

where
R═H, CH₂—CH₂—CH₃, SO₂N(CH₂CH₃)₂, SO₂N(CH₂CH₂CH₃)₂ and
z=1-4, in particular z=1.

Advantages of the compounds according to the invention here may be, in particular:
a surface activity which is equal or superior to conventional hydrocarbon surfactants with respect to efficiency and/or effectiveness,
no degradation to give long-chain persistent fluorocarboxylic or fluorosulfonic acids, such as PFOA (perfluorooctanoic acid) or PFOS (perfluorooctanesulfonate),
relatively low use amounts,
good processability in formulations and/or
storage stability.

The fluorosurfactants according to the invention are particularly suitable for use as hydrophobicising agents or oleophobicising agents.

Areas of use are, for example, the use of the compounds according to the invention as additives in non-aqueous preparations for surface coating, such as radiation-curing and high-solids or low-solids coatings, adhesives, printing inks, paints, surface coatings, photographic coatings, special coatings for semiconductor photolithography (photoresists, top antireflective coatings, bottom antireflective coatings) or in additive preparations for addition to corresponding preparations. For use, the compounds according to the invention are usually introduced into correspondingly designed preparations.

The present invention likewise relates to corresponding compositions which comprise at least one compound according to the invention. Such compositions preferably comprise a carrier which is suitable for the particular application, and optionally further active substances and/or optionally assistants. Preferred compositions here are coatings, printing inks and adhesives. In addition, it is possible for them to be used as interface promoter or emulsifier or viscosity reducer in paints, coatings or adhesives.

The present invention relates secondly to the use of compounds which carry at least one terminal perfluoro-tert-butoxy group and contain at least one substituted or unsubstituted aromatic end group, as surface-active agents.

Preference is given to the use of compounds of the formula (I) or (II). The preferred embodiments thereof are particularly preferred here.

The present invention relates thirdly to a process for the preparation of compounds of the formula (II) comprising the reaction of an alcohol of the formula (IV)

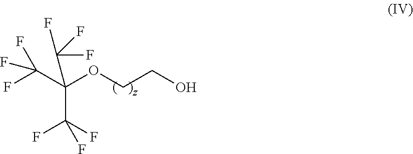

where z=1-30, preferably 1-8,
with an aromatic carboxylic acid R—Ar—COOH or an aromatic carboxylic acid chloride R—Ar—COCl, preferably in the presence of a catalyst. z here is particularly preferably 1-4, in particular z=1.

The synthesis of the alcohol is known from the literature and is described in U.S. Pat. No. 3,385,904.

The compounds according to the invention can preferably be prepared by the following reactions.

1.: Esterification by means of N,N′-dicyclohexylcarbodiimide (DCC) and 4-(dimethylamino)pyridine (DMAP)

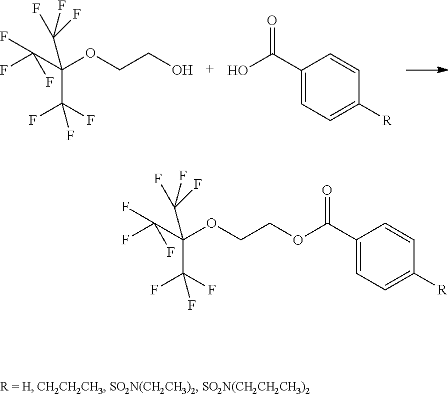

2.: Esterification by means of N-(3-dimethylaminopropyl)-N′-ethylcarbodiimide hydrochloride (EDCl) and 4-(dimethylamino)pyridine (DMAP)

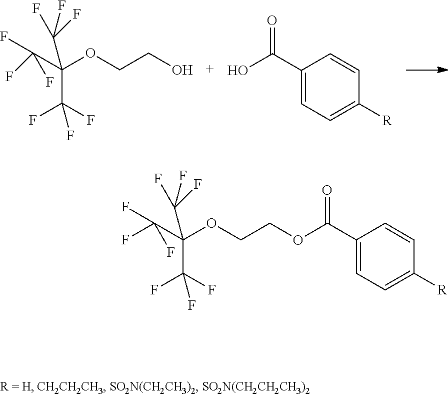

3.: Esterification via the acid chloride

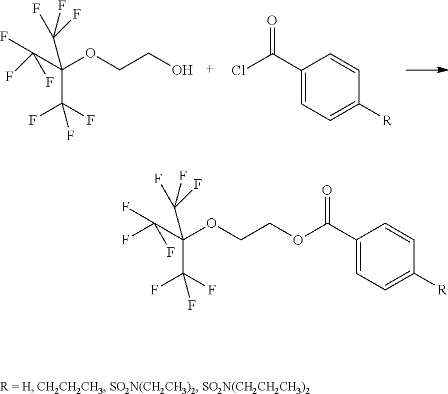

The preparation of further compounds of the formula (II) according to the invention can be carried out analogously to the reactions shown above.

The preparation of further compounds of the formula (I) according to the invention can be carried out by other methods known per se to the person skilled in the art from the literature, as shown below by way of example with reference to some compounds where z=1.

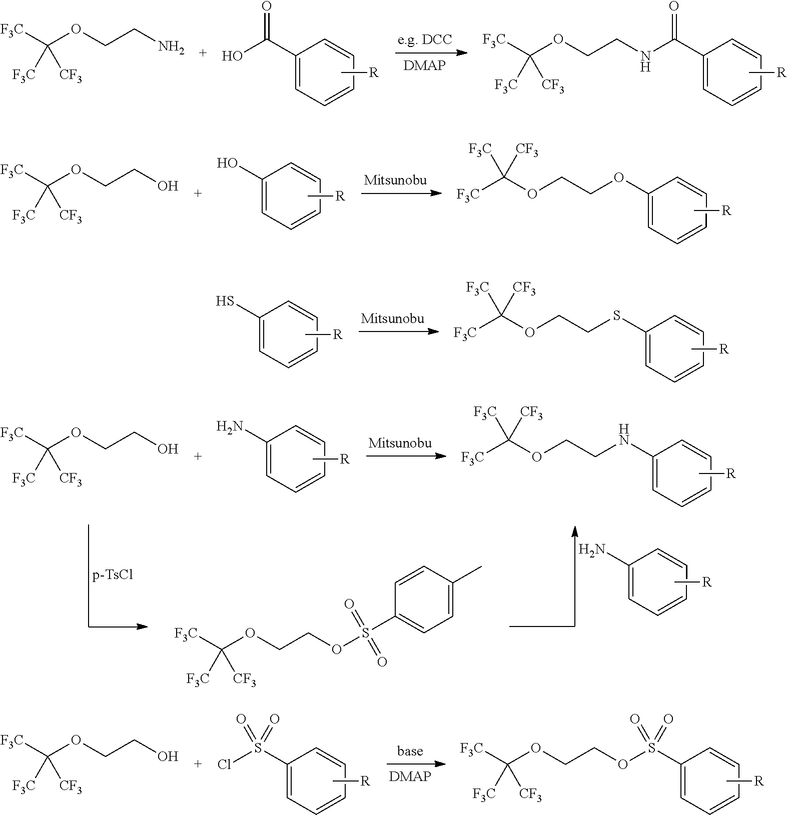

Apart from the preferred compounds mentioned in the description, the use thereof, compositions and processes, further preferred combinations of the subject-matters according to the invention are disclosed in the Claims.

The disclosures in the cited references hereby expressly also belong to the disclosure content of the present application.

The following examples explain the present invention in greater detail without restricting the scope of protection. In particular, the features, properties and advantages described in the examples of the compounds on which the relevant examples are based can also be applied to other substances and compounds which are not mentioned in detail, but fall within the scope of protection, unless otherwise stated elsewhere. In addition, the invention can be carried out throughout the range claimed and is not restricted to the examples given here.

### EXAMPLES

### Example 1

### Esterification by Means of DCC and DMAP

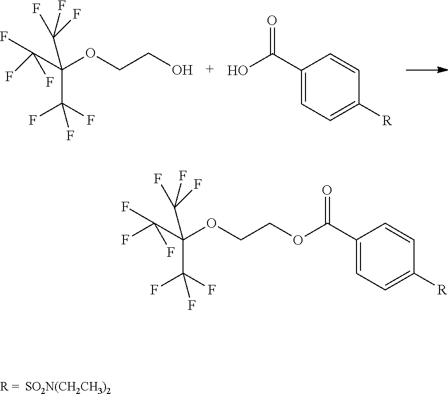

4.91 g (0.0175 mmol) of 2-(perfluoro-tert-butoxy)ethan-1-ol, 5.00 g (0.0175 mmol) of 4-dipropylsulfamoylbenzoic acid and 0.22 g (0.0018 mmol) of 4-(dimethylamino)pyridine (DMAP) in 50 ml of tetrahydrofuran are initially introduced in a round-bottomed flask and cooled to 0° C. A solution of 4.02 g (0.0193 mmol) of N,N′-dicyclohexylcarbodiimide (DCC) in 20 ml of tetrahydrofuran is slowly added dropwise at this temperature. The reaction mixture is warmed to room temperature and stirred for a further 24 hours. The precipitated solid is filtered off, and the filtrate is evaporated in vacuo. The crude product is dissolved in a little ethanol and added to six times the amount of water, whereupon a white solid precipitates. This is filtered off and dried in a drying cabinet.

Substance: C₁₉H₂₂F₉NO₅S M=547.433 g/mol

¹H-NMR (400 MHz; DMSO)

8.12 (d); 7.99 (d); 4.60 (t); 4.48-4.43 (m); 3.08 (t); 1.52-1.42 (m); 0.80 (t) ppm.

¹³C-NMR (100 MHz; DMSO)

163.98; 143.52; 132.16; 129.69; 126.82; 123.75; 120.85; 117.93; 115.02; 68.00; 62.83; 49.00; 20.98; 10.42 ppm.

¹⁹F-NMR (375 MHz; DMSO)

−69.976 ppm.

MS (70 eV) m/e:

547 (M⁺, 5%); 518 (100%).

### Example 2

### Esterification by Means of EDCI and DMAP

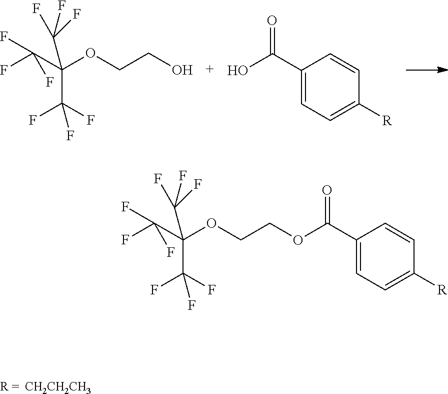

8.80 g (0.0314 mmol) of 2-(perfluoro-tert-butoxy)ethan-1-ol, 5.16 g (0.0314 mmol) of 4-n-propylbenzoic acid and 0.39 g (0.0031 mmol) of 4-(dimethylamino)pyridine (DMAP) in 60 ml of N,N-dimethylformamide are initially introduced in a round-bottomed flask and cooled to 0° C. A solution of 6.69 g (0.0346 mmol) of N-(3-dimethylaminopropyl)-N′-ethylcarbodiimide hydrochloride (EDCl) in 15 ml of N,N-dimethylformamide (+5% of demineralised water) is slowly added dropwise at this temperature. The reaction mixture is warmed to room temperature and stirred for a further 24 hours. The solvent is removed in vacuo.

The crude product is dissolved in a little ethanol and added to six times the amount of 1 N sodium hydroxide solution, whereupon a pale-yellow oil deposits. The water phase is decanted off, and the oil is washed again with water. The oil is dried in vacuo.

Substance: C₁₆H₁₅F₉O₃ M=426.274 g/mol

¹H-NMR (400 MHz; DMSO)

7.87 (d); 7.37 (d); 4.52 (t); 4.45-4.40 (m); 2.63 (t); 1.68-1.56 (m); 0.89 (t) ppm.

¹³C-NMR (100 MHz; DMSO)

164.91; 147.84; 128.54; 128.23; 126.37; 123.77; 120.85; 117.93; 115.03; 68.09; 62.13; 36.60; 23.11; 12.94 ppm.

¹⁹F-NMR (375 MHz; DMSO)

−70.21 ppm.

MS (70 eV) m/e:

426 (M⁺, 22%); 164 (100%).

### Example 3

### Esterification Via the Acid Chloride

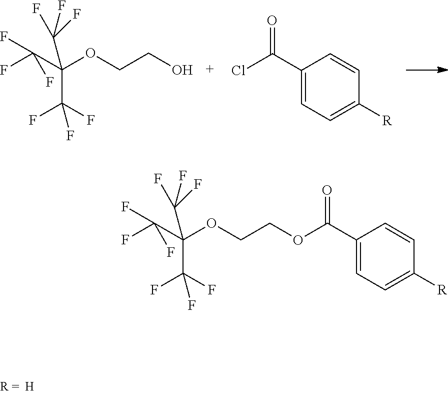

8.80 g (0.0314 mmol) of 2-(perfluoro-tert-butoxy)ethan-1-ol and 4.79 ml (0.0346 mmol) of triethylamine in 90 ml of toluene are initially introduced in a round-bottomed flask and cooled to 0° C. A solution of 4.01 ml (0.0346 mmol) of benzoyl chloride in 20 ml of toluene is slowly added dropwise at this temperature. The reaction mixture is stirred under reflux for 24 hours.

The precipitated solid is filtered off, and the filtrate is evaporated in vacuo. The crude product is dissolved in tert-butyl methyl ether, 1 N sodium hydroxide solution is added, and the phases are separated. The organic phase is washed with water and saturated sodium chloride solution and dried over sodium sulfate.

The solvent is removed in vacuo.

Substance: C₁₃H₉F₉O₃ M=384.194 g/mol

¹H-NMR (400 MHz; DMSO)

7.96 (d); 7.72-7.62 (m); 7.56 (t); 4.55 (t); 4.46-4.42 (m) ppm.

¹³C-NMR (100 MHz; DMSO)

164.93; 161.83; 133.04; 128.75; 128.53; 128.33; 123.78; 120.88; 117.96; 115.03; 68.09; 62.31 ppm.

¹⁹F-NMR (375 MHz; DMSO)

−70.11 ppm.

MS (70 eV) m/e:

384 (M⁺, 4%); 105 (100%).

1. Compounds of the formula (I)
[Yₙ-L-Xₘ]ₒ—Ar (I)
where
Y═(CF₃)₃C—O,
L=linear or branched alkyl having at least 2 C atoms, where one or more non-adjacent C atoms may be replaced by O, S and/or N, and/or one or more double and/or triple bonds may be present in the chain and/or side chain,
X═O(O)C, NH(O)C, O, S, N, OSO₂,
Ar=an unsubstituted or mono- or polysubstituted aromatic ring or condensed ring system having 6 to 18 C atoms, optionally containing N, O and/or S,
n=1-3,
m=0 or 1, and
o=1-3,
where, for o≠1, different L and X may in each case be present and (nonafluoro-tert-butoxy)ethyl tosylate and di(perfluoro-tert-butoxyethyl) phthalate are excluded.

2. Compounds according to claim 1, characterised in that Ar=an aromatic ring or condensed ring system which is mono- or polysubstituted by halogen, alkyl, cycloalkyl, aryl, —OH, —OR¹, —NR¹R², —C(O)OH, —COOR¹, —CONR¹R² or —SO₂NR¹R², where R¹ and R²=independently of one another H, linear or branched alkyl, cycloalkyl or alkylaryl.

3. Compounds according to claim 1, characterised in that X═O(O)C.

4. Compounds according to claim 1, characterised in that L=linear alkyl.

5. Compounds of the formula (II) 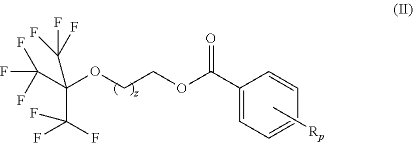
where
z=1-30,
R═H, C1-C8-alkyl, —OH, —NR¹R² or —SO₂NR¹R²,
R¹ and R²=independently of one another H, C1-C8-alkyl and
p=0-3.

6. Compounds of the formula (III) 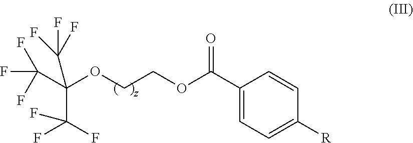
where
R═H, CH₂—CH₂—CH₃, SO₂N(CH₂CH₃)₂, SO₂N(CH₂CH₂CH₃)₂ and
z=1.

7. A method comprising applying a surface coating to a surface, wherein said surface coating contains at least one perfluoro-tert-butoxy group and at least one substituted or unsubstituted aromatic end group as surface-active agent.

8. A method according to claim 7, characterised in that said surface active agent is a compound of formula (I), (II) or (III):
[Yₙ-L-Xₘ]ₒ—Ar (I)
where
Y═(CF₃)₃C—O,
L=linear or branched alkyl having at least 2 C atoms, where one or more non-adjacent C atoms may be replaced by O, S and/or N, and/or one or more double and/or triple bonds may be present in the chain and/or side chain,
X═O(O)C, NH(O)C, O, S, N, OSO₂,
Ar=an unsubstituted or mono- or polysubstituted aromatic ring or condensed ring system having 6 to 18 C atoms, optionally containing N, O and/or S,
n=1-3,
m=0 or 1, and
o=1-3,
where, for o≠1, different L and X may in each case be present and (nonafluoro-tert-butoxy)ethyl tosylate and di(perfluoro-tert-butoxyethyl) phthalate are excluded; 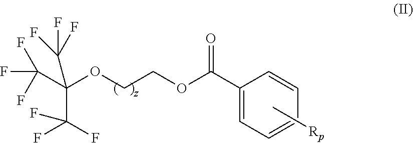
where
z=1-30,
R═H, C1-C8-alkyl, —OH, —OR¹, —NR¹R² or —SO₂NR¹R²,
R¹ and R²=independently of one another H, C1-C8-alkyl and
p=0-3; and 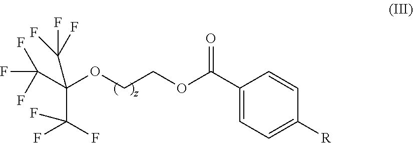
where
R═H, CH₂—CH₂—CH₃, SO₂N(CH₂CH₃)₂, SO₂N(CH₂CH₂CH₃)₂ and
z=1.

9. Process for the preparation of compounds of the formula (II) comprising the reaction of an alcohol of the formula (IV) 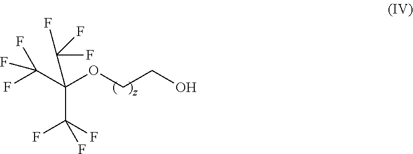
where z=1-30,
with an aromatic carboxylic acid R—Ar—COOH or an aromatic carboxylic acid chloride R—Ar—COCl, preferably in the presence of a catalyst.

10. Process according to claim 9, characterised in that the reaction is carried out in the presence of a) N,N′-dicyclohexylcarbodiimide or N-(3-dimethylaminopropyl)-N′-ethylcarbodiimide hydrochloride and b) 4-(dimethylamino)pyridine.

11. A composition comprising at least one compound according to claim 1, a carrier, and optionally further active substances and/or optionally assistants.

12. A composition comprising at least one compound according to claim 5, a carrier, and optionally further active substances and/or optionally assistants.

13. A composition comprising at least one compound according to claim 6, a carrier, and optionally further active substances and/or optionally assistants.
